(19) United States
(12) Patent Application Publication
(10) Pub. No.: US 2017/0156999 A1
(43) Pub. Date: Jun. 8, 2017

(54) PERSONAL CARE COMPOSITION IN A DISSOLVABLE CONTAINER

(71) Applicant: The Dial Corporation, Scottsdale, AZ (US)

(72) Inventors: Jennifer Harris, Phoenix, AZ (US); James A. Fernandez, Phoenix, AZ (US)

(21) Appl. No.: 14/957,894

(22) Filed: Dec. 3, 2015

(51) Int. Cl. A61K 8/11; A45D 40/00; A61K 8/81; A61K 8/92; A61Q 19/10; A61Q 19/00

(52) U.S. Cl. CPC A61K 8/11; A61Q 19/10; A61Q 19/007; A61K 8/8129; A61K 8/8135; A61K 8/92; A45D 40/00; A61K 2800/28; A61K 2800/596

(57) ABSTRACT

The present disclosure describes a cleansing device. The cleansing device includes a container that is dissolvable in water. The cleansing device also includes a personal care composition. The personal care composition is disposed within the container. When the container dissolves, the personal care composition is to be deposited on a surface.

### FIELD OF THE INVENTION

The present invention generally relates to a cleansing device, and more particularly relates to a device that includes a dissolvable container that dispenses the composition once dissolved.

### BACKGROUND OF THE INVENTION

Personal care compositions are a part of most people's lives with such compositions being used daily, if not more often. Some personal care compositions are stored in containers that are difficult and/or messy to use. For example, body scrubs and hand scrubs are often provided in large open-mouthed jars or bottle. When used in some environments, these type of containers can create a mess. Moreover when using such containers, a portion of the composition is often wasted, as it is unreachable, or too difficult to reach by a user.

For example, when used in a shower, these containers allow water and other contaminants, such as bacteria, to come into contact with the composition, often for long periods of time after an individual has ended the shower. As the contaminants and/or water enter the container, the composition therein may be unusable or more difficult to use. Still further, the contaminants, as suggested by the term, contaminate the composition, making it unfit for its intended use. Still further, the water can dilute the composition or otherwise break it down such that it is unusable, unstable, or less effective when used. Yet another complication with containers of some personal care compositions is that they may inadvertently spill out of the container, and onto other fabrics, thus tarnishing those fabrics or surfaces creating a mess. For example, when placed in a bag, a container may open spilling the contents of the container onto other surfaces such as the interior of a purse.

Accordingly, it is desirable to have a cleansing device that prevents contamination of a personal care composition which contamination threatens the efficacy of the product and may make the product more difficult to apply. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cleansing device is described. The cleansing device includes a container that is dissolvable in water. The cleansing device also includes a personal care composition disposed within the container. When the container dissolves, the personal care composition is deposited on a surface.

The present application also describes a cleansing device that includes a polyvinyl alcohol container that is dissolvable when in contact with water. The device also includes a personal care composition disposed within the polyvinyl alcohol container. The personal care composition is deposited on skin when the polyvinyl alcohol container dissolves. The personal care composition includes a moisturizer, a humectant, an abrasive, a cleanser and a dispersing agent.

The present disclosure also describes a personal care consumer product. The personal care consumer product includes a housing to hold multiple personal care devices and a number of personal care devices disposed within the housing. A personal care device includes an enclosed film dissolvable in water and a personal care composition disposed within the enclosed film. The personal care composition is to be deposited on a surface when the enclosed film dissolves.

### BRIEF DESCRIPTION OF THE DRAWINGS

The present invention will hereinafter be described in conjunction with the following drawing figures, wherein like numerals denote like elements, and

FIG. 1 is a diagram of a dissolvable container with a personal care composition disposed therein according to the principles described herein.

FIG. 2 is a cross-sectional diagram of a dissolvable container with a personal care composition disposed therein according to the principles described herein.

FIG. 3 is a diagram of a personal care consumer product that includes multiple personal care devices according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, compositions, and personal care compositions in particular, are commonly used by most individuals. Such compositions are applied regularly; for example every day, with some compositions being applied multiple times per day. If contaminants come into contact with such compositions, the efficacy of the composition may suffer. Moreover, water that comes into contact with such compositions, that is left to sit for long periods of time also dilute or otherwise degrade the composition and also affect its performance. Such contamination can occur as a user scoops the composition out of the container and bio contaminants, such as bacteria, from the user and/or water from a shower enter, and contaminate the composition. Moreover, due to the limitations of current containers, much of the product is left unused which could result in lost revenue for a producer and frustration by the consumer as they are not using all the product they purchased.

Accordingly, the principles described herein provide a cleansing device that includes a dissolvable container that includes a personal care composition inside. As the container comes into contact with water and dissolves, the personal care composition inside is free to be applied to a user's skin. The composition may be a body scrub, or any other composition that does not react with the dissolvable film from the inside.

The cleansing device disclosed herein may improve the efficacy of a cleansing composition by preventing contamination of the composition from contaminants such as bio contaminants by a user or from entry of water into the composition before or during its intended use. Moreover the cleansing device disclosed herein facilitates the full use of the purchased amount.

As used in the present specification and in the appended claims, the term “a number of” or similar language may include any positive number, including one to infinity; zero not being a number, but the absence of a number.

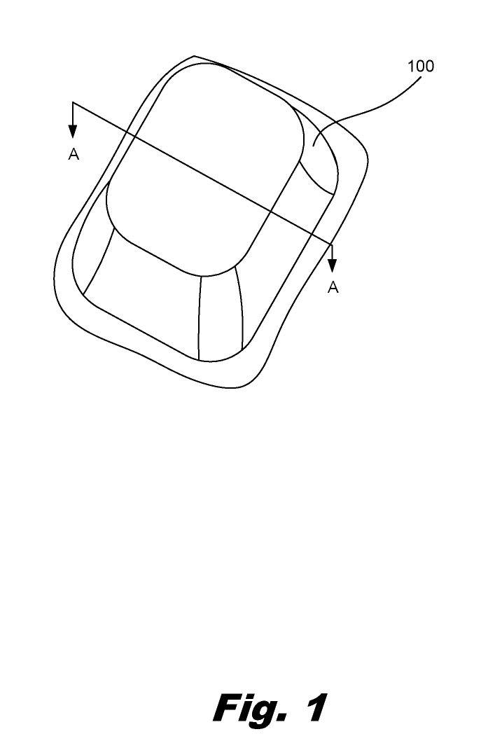

Turning now to the figures, FIG. 1 is a diagram of an example of a dissolvable container (100) with a personal care composition disposed therein according to the principles described herein. As described above, the container (100) is dissolvable in water, meaning that when the container (100) comes into contact with sufficient quantities of water, the container (100) breaks down. When the container (100) breaks down, the contents within the container (100) are available to be applied to the skin. In other words, the personal care composition that is disposed within the container (100) can be deposited on the skin once the container (100) has dissolved. While FIG. 1 depicts the container (100) as having a particular size and shape, the container (100) may be any size and shape. For example, the container (100) may be of a size to hold a single-use quantity of the personal care composition, which single-use quantity is determined by the personal care composition deposited therein.

One example of a dissolvable container (100) is a container that is formed of polyvinyl alcohol. Specifically, the polyvinyl alcohol is in a film form and shaped to make the container (100). The personal care composition is disposed therein and the film sealed to form an enclosed container (100). Another example of a dissolvable container (100) material is polyvinyl acetate, again which may a film that is formed into a device that can contain a composition. While specific reference is made to polyvinyl alcohol and polyvinyl acetate, any material may be used for the container (100) so long as it is dissolvable in the presence of water such that the contents of the container (100) may be allowed to contact the skin surface.

In addition to being formed of different materials, the container (100) may be different colors. For example, the container (100) may be transparent, or may have colorant added. The container (100) may therefore, be opaque, translucent, or transparent, and may or may not have colorant added thereto.

Using a dissolvable container (100) as disclosed herein may be beneficial in that it protects the composition from exposure to contaminants and the surrounding environment. For example, in a shower after use, errant water from the shower may contact a composition that is in an open-mouthed jar. After the shower, the water may remain and break down the abrasive material in the composition such that it loses its efficacy.

Still further using single-size containers (100) may allow for portability of the composition. For example, one or multiple single-size containers (100) may be stored in a bag or purse, and a user can easily transport the composition to various locations.

Even further, using such dissolving containers (100) ensures that all of a purchased product is used. For example, as described above, when contained in a bottle, it is difficult, if possible, to consume all the composition disposed therein. With such dissolving containers (100) all the product is cleanly stored and used.

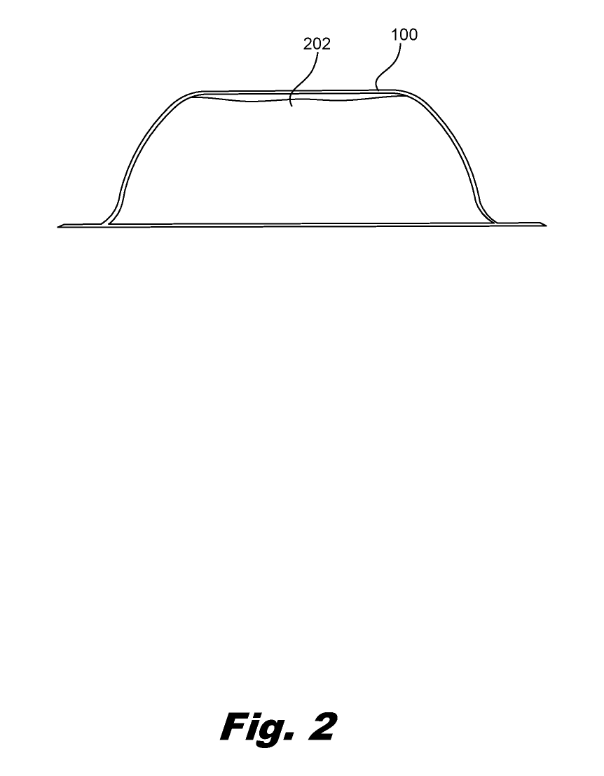

FIG. 2 is a cross-sectional diagram of a dissolvable container (100) with a personal care composition (202) disposed therein according to the principles described herein. More specifically, FIG. 2 is a cross-sectional diagram taken along the line “A” in FIG. 1. FIG. 2 clearly indicates the film nature that the container (100) may have.

As described above, the container (100) includes a personal care composition (202). A personal care composition (202) may be used for various purposes and may take various forms. For example, the personal care composition (202) may be intended to be applied to skin. Examples, of personal care compositions (202) that are applied to skin include, but are not limited to, a body scrub, a hand scrub, a foot scrub, a face scrub, an acne treatment, a soap, and a lotion. The personal care composition (202) may have an oil as its base ingredient as opposed to water. Such a personal care composition (202) is beneficial as it does not include sufficient quantities of water to interact with, and breakdown the dissolvable container (100).

In some examples, the personal composition (202) may not have a form when not included in a container (100). In other words, the composition (202) may be a gel composition (202) or a fluid composition (202). Another example of a composition (202) without form is a powder composition (202). In this example, the powder may be compressed and packed into a polyvinyl alcohol or polyvinyl acetate container (100). When the container (100) dissolves, the composition (202) may be applied to the surface such as skin.

The personal care composition (202) includes multiple ingredients to carry out a particular function. In one example the personal care composition (202) is a skin scrub that is used to remove dead or dying skin particles from the body to reveal healthier looking skin. Examples of skin scrubs include, but are not limited to a body scrub, a foot scrub, a hand scrub, and a face scrub. Such a skin scrub personal care composition (202) may include a moisturizer, a humectant, an abrasive, a cleanser, and a dispersing agent.

The moisturizer is a component that makes the external layers of the skin and epidermis softer and more pliable. This is accomplished by retaining the skin's water content. The moisturizer of the personal care composition (202) may also serve as a carrier fluid to act as a carrier for the other ingredients in the composition. The moisturizer may form 10-30 percent by weight of the personal care composition (202). The carrier fluid may be an oil so as to form the basis of a personal care composition (202).

Examples of oils that may be used as a moisturizer/carrier fluid include, but are not limited to, abies alba seed oil, acacia victoriae seed oil, acanthosicyos horridus seed oil, acetic/linoleic/palmitic triglyceride, acetylated castor oil, acetylated hydrogenated castor oil, acrocomia aculeata seed butter, actinidia chinensis (kiwi) seed oil, adansonia digitata oil, adansonia digitata seed oil, adeps bovis, adeps suillus, akebia quinata seed oil, aleurites moluccanus bakoly seed oil, aleurites moluccanus seed oil, allium sativum (garlic) bulb oil, amaranthus caudatus seed oil, amaranthus cruentus seed oil, amaranthus hypochondriacus seed oil, amaranthus spinosus seed oil, amburana cearensis seed oil, anacardium occidentale (cashew) seed oil, arachis hypogaea (peanut) oil, arctium lappa seed oil, argania spinosa kernel oil, argemone mexicana seed oil, astrocaryum murumuru seed butter, astrocaryum tucuma seed butter, astrocaryum tucuma seed oil, astrocaryum vulgare fruit oil, astrocaryum vulgare kernel oil, attalea maripa seed butter, aurantiochytrium limacinum oil, avena sativa (oat) kernel oil, avena sativa (oat) sprout oil, badger oil, baillonella toxisperma seed oil, balanites aegyptiaca kernel oil, balanites roxburghii seed oil, banksia serrata seed oil, barklya syringifolia seed oil, bassia butyracea seed butter, bassia latifolia seed butter, bear oil, bertholletia excelsa seed oil, bixa orellana seed oil, black sea rod oil, bombyx lipida, borago officinalis seed oil, brain lipids, brassica campestris (rapeseed) seed oil, brassica napus seed oil, brassica oleracea acephala seed oil, brassica oleracea italica (broccoli) seed oil, brevoortia oil, bubulum oil, buffalo fat, buglossoides arvensis seed oil, butter, butter extract, butyrospermum parkii (shea) butter, butyrospermum parkii (shea) oil, butyrum, c8-12 acid triglyceride, c12-18 acid triglyceride, calendula officinalis seed oil, callitris columellaris seed oil, calodendrum capense nut oil, calophyllum inophyllum seed oil, calophyllum tacamahaca seed oil, camellia chekiangoleosa seed oil, camellia japonica seed oil, camellia kissi seed oil, camellia oleifera seed oil, camellia reticulata seed butter, camellia reticulata seed oil, camellia seed oil, camellia sinensis seed oil, canarium indicum seed oil, cannabis sativa seed oil, canola oil, caprae butyrum, capric/lauric/myristic/oleic triglyceride, caprylic/capric/lauric triglyceride, caprylic/capric/linoleic triglyceride, caprylic/capric/myristic/stearic triglyceride, caprylic/capric/palmitic/stearic triglyceride, caprylic/capric/stearic triglyceride, caprylic/capric triglyceride, carapa guaianensis seed oil, carapa procera seed oil, carica papaya (papaya) seed oil, carpotroche brasiliensis seed butter, carpotroche brasiliensis seed oil, carthamus tinctorius (safflower) seed oil, carya illinoinensis (pecan) seed oil, caryocar brasiliense fruit oil, caryocar coriaceum seed oil, caryocar villosum kernel oil, caryodendron orinocense seed oil, castoryl maleate, caviar oil, cedrus deodara seed oil, cephalins, chaenomeles japonica seed oil, chenopodium quinoa seed oil, chlorella protothecoides oil, cibotium barometz oil, c10-40 isoalkyl acid triglyceride, citrullus lanatus (watermelon) seed oil, citrus aurantifolia (lime) seed oil, citrus aurantium dulcis (orange) seed oil, citrus australasica seed oil, citrus depressa seed oil, citrus glauca seed oil, citrus grandis (grapefruit) seed oil, citrus iyo oil, citrus junos seed oil, citrus limon (lemon) seed oil, citrus paradisi (grapefruit) seed oil, citrus sunki seed oil, cocos nucifera (coconut) oil, cocos nucifera (coconut) seed butter, cod liver/mink/tallow triglyceride, cod liver oil, coffea arabica (coffee) seed oil, coix lacryma-jobi (job's tears) seed oil, corylus americana (hazelnut) seed oil, corylus avellana (hazelnut) seed oil, crambe abyssinica seed oil, crocodile oil, c10-18 triglycerides, cucumis melo (melon) seed oil, cucumis sativus (cucumber) seed oil, cucurbita pepo (pumpkin) seed oil, cuphea lanceolata/viscosissima seed oil, cuphea viscosissima seed oil, cynara cardunculus seed oil, davidsonia jerseyana seed oil, deer fat, dipteryx odorata seed butter, docosahexenoic/docosapentenoic/oleic/palmitic triglyceride, donkey oil, dracocephalum moldavica seed oil, dromiceius oil, echinops sphaerocephalus seed oil, echium lycopsis fruit oil, echium plantagineum seed oil, egg oil, elaeagnus mollis seed oil, elaeis guineensis (palm) butter, elaeis guineensis (palm) kernel oil, elaeis guineensis (palm) oil, elaeis oleifera fruit oil, elaeis oleifera kernel oil, elaeis (palm) fruit oil, emu oil, epoxidized soybean oil, euphorbia lathyris seed oil, euterpe oleracea fruit oil, fish liver oil, fish oil, fragaria ananassa (strawberry) seed oil, fragaria chiloensis (strawberry) seed oil, fragaria vesca (strawberry) seed butter, fragaria vesca (strawberry) seed oil, fragaria virginiana (strawberry) seed oil, fusanus spicatus kernel oil, gadi iecur oil, garcinia indica seed butter, gevuina avellana oil, gevuina avellana seed oil, glyceryl triacetyl hydroxystearate, glyceryl triacetyl ricinoleate, glyceryl tri-hydrogenated rosinate, glycine max (soybean) oil, glycine soja (soybean) lipids, glycine soja (soybean) oil, goat butter, goat fat, gossypium herbaceum (cotton) seed oil, gossypium hirsutum (cotton) seed oil, guizotia abyssinica seed oil, haematococcus pluvialis oil, halorubrum tebenquichense lipids, helianthus annuus (sunflower) seed butter, helianthus annuus (sunflower) seed oil, hibiscus sabdariffa seed oil, hippophae rhamnoides fruit oil, hippophae rhamnoides oil, hippophae rhamnoides seed oil, hoplostethus oil, horse fat, human placental lipids, hydnocarpus pentandrus kernel oil, hydrogenated adansonia digitata seed oil, hydrogenated apricot kernel oil, hydrogenated argania spinosa kernel oil, hydrogenated black currant seed oil, hydrogenated calophyllum inophyllum seed oil, hydrogenated camelina sativa seed oil, hydrogenated camellia japonica seed oil, hydrogenated camellia oleifera seed oil, hydrogenated canola oil, hydrogenated castor oil, hydrogenated castor oil laurate, hydrogenated castor oil triisostearate, hydrogenated coconut oil, hydrogenated corn oil, hydrogenated cottonseed oil, hydrogenated crambe abyssinica seed oil, hydrogenated cranberry seed oil, hydrogenated c12-18 triglycerides, hydrogenated egg oil, hydrogenated evening primrose oil, hydrogenated fish oil, hydrogenated grapefruit seed oil, hydrogenated grapeseed oil, hydrogenated hazelnut oil, hydrogenated hemp seed oil, hydrogenated horse fat, hydrogenated kukui nut oil, hydrogenated lard, hydrogenated lime seed oil, hydrogenated macadamia seed oil, hydrogenated meadowfoam seed oil, hydrogenated menhaden oil, hydrogenated milk lipids, hydrogenated mink oil, hydrogenated moringa oleifera seed oil, hydrogenated olive oil, hydrogenated orange roughy oil, hydrogenated orange seed oil, hydrogenated ostrich oil, hydrogenated palm kernel oil, hydrogenated palm oil, hydrogenated panax ginseng root oil, hydrogenated passiflora edulis seed oil, hydrogenated peach kernel oil, hydrogenated peanut oil, hydrogenated pinus pinaster seed oil, hydrogenated pistachio seed oil, hydrogenated pistacia vera seed oil, hydrogenated plukenetia volubilis seed oil, hydrogenated prunus domestica seed oil, hydrogenated pumpkin seed oil, hydrogenated punica granatum seed oil, hydrogenated rapeseed oil, hydrogenated raspberry seed oil, hydrogenated rice bran oil, hydrogenated rosa canina fruit oil, hydrogenated safflower seed oil, hydrogenated sesame seed oil, hydrogenated shark liver oil, hydrogenated shea butter, hydrogenated shea oil, hydrogenated soybean oil, hydrogenated sunflower seed oil, hydrogenated sweet almond oil, hydrogenated tallow, hydrogenated vegetable oil, hydrogenated walnut seed oil, hydrogenated wheat germ oil, hydrolyzed theobroma cacao (cocoa) seed butter, hydroxy/methoxy soybean oil, illipe butter, irvingia gabonensis kernel butter, isatis tinctoria seed oil, isomerized palm oil, juglans regia (walnut) seed butter, juglans regia (walnut) seed oil, kniphofia uvaria seed oil, kyounin yu, lactis lipida, lagenaria sphaerica seed oil, lappula squarrosa seed oil, lard, lauric/palmitic/oleic triglyceride, laurus nobilis fruit oil, lecythis minor seed oil, lesquerella fendleri seed oil, limnanthes alba (meadowfoam) seed oil, linum usitatissimum (linseed) seed oil, liriodendron tulipifera leaf wax, lithospermum officinale seed oil, luffa cylindrica seed oil, lupinus albus seed oil, lycium barbarum seed oil, macadamia integrifolia seed oil, macadamia ternifolia seed oil, machilus thunbergii seed oil, maleated soybean oil, mallotus japonicus seed oil, mamushi oil, mangifera indica (mango) seed butter, mangifera indica (mango) seed oil, mare milk lipids, marmot oil, marrow lipids, mauritia flexuosa fruit oil, melia azadirachta seed oil, melissa officinalis seed oil, menhaden oil, milk lipids, mink oil, momordica cochinchinensis seed aril oil, moray eel oil, morinda citrifolia seed oil, moringa oleifera seed oil, mortierella oil, mucor circinelloides oil, mustela oil, mustelic/palmitic triglyceride, nacre lipids, natane yu, neatsfoot oil, nelumbium speciosum flower oil, nelumbo nucifera seed oil, nigella sativa seed oil, odontella aurita oil, oenocarpus bacaba fruit oil, oenocarpus bataua fruit oil, oenocarpus bataua seed oil, oenothera biennis (evening primrose) oil, olea europaea (olive) fruit oil, olea europaea (olive) husk oil, oleic/linoleic triglyceride, oleic/palmitic/lauric/myristic/linoleic triglyceride, oleostearine, olus oil, omental lipids, opuntia ficus-indica seed oil, opuntia stricta seed oil, orange roughy oil, orbignya cohune seed oil, orbignya martiana seed oil, orbignya oleifera seed oil, orbignya speciosa kernel oil, oryza sativa (rice) bran oil, oryza sativa (rice) germ oil, oryza sativa (rice) lipids, oryza sativa (rice) seed oil, ostrich oil, ovum oil, oxidized corn oil, oxidized hazelnut seed oil, oxidized pogostemon cablin leaf oil extract, oxycoccus palustris seed oil, paeonia rockii seed oil, paeonia suffruticosa seed oil, palmitic/stearic triglyceride, panax ginseng seed oil, panicum miliaceum (millet) seed oil, papaver orientale (poppy) seed oil, papaver somniferum seed oil, parinari curatellifolia seed oil, passiflora edulis seed oil, pentaclethra macroloba seed oil, perilla ocymoides seed oil, persea gratissima (avocado) butter, persea gratissima (avocado) fruit butter, persea gratissima (avocado) oil, phormium tenax seed oil, pinus densiflora seed oil, pinus halepensis seed oil, pinus koraiensis seed oil, pinus parviflora seed oil, pinus pentaphylla seed oil, pinus pinaster seed oil, pinus sibirica seed oil, piper nigrum (pepper) seed oil, piscum lecur oil, pistacia vera seed oil, placental lipids, plukenetia volubilis seed oil, pollen lipids, pongamia glabra seed oil, pouteria sapota seed butter, prinsepia utilis seed oil, prunus amygdalus dulcis (sweet almond) oil, prunus armeniaca (apricot) kernel oil, prunus avium (sweet cherry) seed oil, prunus cerasus (bitter cherry) seed oil, prunus domestica seed oil, prunus insititia seed oil, prunus pedunculata seed oil, prunus persica (peach) kernel oil, psidium guajava seed oil, punica granatum seed oil, pycnanthus angolensis nut oil, pyrus cydonia seed oil, pyrus malus (apple) seed oil, rabbit fat, raphanus sativus (radish) seed oil, ribes nigrum (black currant) seed oil, ribes rubrum (currant) seed oil, ricinodendron rautenenii seed oil, ricinoleic/caproic/caprylic/capric triglyceride, ricinus communis (castor) seed oil, rosa canina fruit oil, rosa eglantaria seed oil, rosa moschata seed oil, rosa multiflora seed oil, rosa roxburghii seed oil, rosa rubiginosa seed oil, rubus chamaemorus seed oil, rubus glaucus seed oil, rubus idaeus (raspberry) seed oil, rubus laciniatus seed oil, salmon oil, salmo oil, salvia hispanica seed oil, salvia sclarea (clary) seed oil, sambucus nigra seed oil, santalum album (sandalwood) seed oil, santalum spicatum seed oil, schinus molle oil, schinus terebinthifolia seed oil, schinziophyton rautanenii kernel oil, schleichera trijuga seed oil, sclerocarya birrea seed oil, sea snake lipids, serenoa serrulata fruit oil, sesamum indicum (sesame) seed butter, sesamum indicum (sesame) seed oil, shark lipids, shark liver oil, shorea robusta seed butter, shorea stenoptera seed butter, silkworm chrysalis oil, silkworm lipids, silybum marianum seed oil, skin lipids, solanum lycopersicum (tomato) fruit lipids, solanum lycopersicum (tomato) fruit oil, solanum lycopersicum (tomato) seed oil, sorbus aucuparia seed oil, soybean glycerides, spinal cord lipids, sterculia africana seed oil, strychnos potatorum seed oil, synsepalum dulcificum seed oil, tall oil, tallol, tallow, taraktogenos kurzii seed oil, telfairia pedata oil, terminalia ferdinandiana seed oil, theobroma cacao (cocoa) seed butter, theobroma grandiflorum seed butter, torreya nucifera seed oil, triarachidin, tribehenin, tricaprin, tricaprylin, trichilia emetica seed butter, trichilia roka seed butter, trichodesma zeylanicum oil, trierucin, triethylhexanoin, triheptanoin, triheptylundecanoin, trihydroxymethoxystearin, trihydroxystearin, triisononanoin, triisopalmitin, triisostearin, trilaurin, trilinolein, trilinolenin, trimyristin, triolein, tripalmitin, tripalmitolein, triricinolein, trisebacin, tristearin, triticum aestivum (wheat) flour lipids, triticum aestivum (wheat) germ oil, triticum vulgare (wheat) bran lipids, triticum vulgare (wheat) flour lipids, triticum vulgare (wheat) germ oil, triundecanoin, turtle oil, ulkenia amoeboidea oil, vaccinium corymbosum (blueberry) seed oil, vaccinium macrocarpon (cranberry) seed oil, vaccinium myrtillus seed oil, vaccinium vitis-idaea seed oil, vateria indica seed butter, vegetable oil, vigna radiata seed oil, virola sebifera nut oil, vitis vinifera (grape) seed oil, whale oil, xanthoceras sorbifolium seed oil, ximenia americana seed oil, zea mays (corn) germ oil, and zea mays (corn) oil.

The personal care composition (202) also includes a humectant which is a hygroscopic molecule with hydrophilic groups attached thereto. A humectant forms a hydrogen bond with molecules of water, thus increasing the water content of the upper layers of the skin. The humectant in some cases serves as a secondary carrier fluid. which may form between 20-30 percent by weight of the personal care composition (202).

Examples of humectants include, but are not limited to glycerin, abies homolepis leaf extract, abies veitchii leaf extract, acacia victoriae seed oil, acetyl arginine, acetyl hydroxyproline, adansonia digitata pulp extract, adansonia grandidieri seed oil, adenophora stricta root extract, agave atrovirens extract, alanyl glutamine, alcaligenes polysaccharides, algae extract (retired), alnus japonica leaf/stem water, aloe barbadensis leaf extract, aloe barbadensis leaf polysaccharides, aloe yohjyu matsu ekisu, alpinia speciosa leaf oil, amidinoproline, ananas erectifolius leaf juice, angelica acutiloba root/citrus aurantium amara (bitter orange) fruit/coptis japonica root/forsythia viridissima fruit/gardenia florida fruit/glycyrrhiza glabra (licorice) root/mentha arvensis leaf/paeonia lactiflora root/platycodon grandiflorus root/rehmannia glutinosa root/scutellaria baicalensis root extract, angelica furcijuga flower/leaf/stem extract, anhydrogalactose, anhydroglucitol, annona atemoya fruit extract, aphanizomenon flos-aquae extract, arachis hypogaea (peanut) sprout extract, araucaria angustifolia seedcoat extract, argania spinosa seedcake extract, arthraxon hispidus flower/leaf/stem extract, aspergillus/amaranthus hypochondriacus seed ferment filtrate, aspergillus ferment extract filtrate, aspergillus/soybean seed extract ferment extract filtrate, aspergillus/soybean/wheat germ/camellia sinensis leaf/job's tears seed/rice germ/sesame seed ferment, aspergillus/soybean/wheat germ/camellia sinensis leaf/job's tears seed/rice germ/sesame seed ferment extract, auricularia polytricha fruiting body extract, bacillus/rice bran extract/soybean extract ferment filtrate, bamboo vinegar, banksia serrata seed oil, barklya syringifolia flower/leaf extract, barklya syringifolia seed oil, behenoxy peg-10 dimethicone, benzyl pca, betaine, biota orientalis leaf extract, bis-glyceryl ascorbate, bis-hydroxylauryl dimethicone/ipdi copolymer, bis-isobutyl peg-24/ppg-7/dimethicone copolymer, bis-(methoxy peg-100 polyepsilon caprolactone) sebacate, bis-palmitoyl tromethamine, bis-peg/ppg-15/5 dimethicone, bis-phenylisopropyl phenylisopropyl dimethicone/vinyl dimethicone crosspolymer, bittern, black strap powder, botryococcus braunii extract, bromoethyl pca, 2,3-butanediol, butyl 2-glyceryl ascorbate, butyl 3-glyceryl ascorbate, sec-butylphenyl ethyl glyceryl diether, t-butylphenyl ethyl glyceryl diether, sec-butylphenyl glyceryl ether, t-butylphenyl glyceryl ether, calcium phosphoryl oligosaccharides, c12-13 alkyl glyceryl hydrolyzed hyaluronate, callitris columellaris leaf/wood extract, callitris columellaris seed oil, caprylyl 2-glyceryl ascorbate, caprylyl 3-glyceryl ascorbate, caprylyl glycol/glycerin/polyacrylic acid copolymer, capsaicinyl glucosides, carboxymethyl caprooyl chitosan, carnitine hcl, caryocar brasiliense fruit/leaf extract, caulerpa okamurae extract, cercis chinensis flower/leaf/stem extract, cetyl 2-glyceryl ascorbate, cetyl 3-glyceryl ascorbate, chitosan hydroxypropyltrimonium chloride, chitosan lauroyl glycinate, chloroethyl pca, cholesterol/hdi/pullulan copolymer, cichorium intybus (chicory) seed oil, cimicifuga dahurica root/forsythia virdissima fruit/glycyrrhiza glabra (licorice) root/paeonia lactiflora root/platycodon grandiflorus root/pueraria lobata root extract, citrus australasica fruit extract, citrus australasica seed oil, citrus glauca seed oil, citrus jabara pericarp extract, citrus sunki peel extract, citrus tankan fruit water, cladosiphon novae-caledoniae extract, clay minerals, cobalt dna, coix lacryma-jobi ma-yuen shell extract, coleus forskohlii root extract, colloidal gold/camellia sinensis leaf powder, commelina communis flower/leaf/stem extract, cordyceps militaris (mushroom) ferment filtrate, crosslinked bacillus/glucose/sodium glutamate ferment, cryptomeria japonica leaf extract, cucumis melo (melon) fruit water, curcuma aromatica leaf extract, curcuma zedoaria leaf extract, cyclohexylglycerin, cyclopia intermedia leaf extract, cymbidium erythraeum flower extract, daedalea dickinsii mycelium ferment filtrate, diglycereth-7 malate, diglycerin, diglycol guanidine succinate, dihydroxypropyl arginine hcl, diisotridecyl lauroyl glutamate, dilauroyl 3-glyceryl ascorbate, dimethicone/divinyldimethicone/silsesquioxane crosspolymer, dimyristoyl glyceryl ascorbate, dipentaerythrityl tetrabehenate/polyhydroxystearate, distearoyl glyceryl ascorbate, dunaliella bardawil extract, earthworm conditioned soil, earthworm conditioned soil extract, ecklonia cava extract, enterococcus faecalis, epilobium parviflorum leaf/stem extract, eremophila longifolia seed oil, erythritol, ethylhexyl hydroxystearoyl hydroxystearate, flindersia acuminata leaf extract, foeniculum vulgare (fennel) leaf extract, fructose, ganoderma lucidum (mushroom) ferment filtrate, gluconobacter ferment filtrate, glucose, glucosyl hesperidin, glucosyl naringin, glucuronolactone, glycereth-7 glycolate, glycerylamidoethyl methacrylate/stearyl methacrylate copolymer, glyceryl ascorbate, 3-glyceryl ascorbate, glyceryl citrate crosspolymer, glyceryl dimaltodextrin, glyceryl myristyl lauroyl glutamate, glyceryl olivate oleate estolides, glycine max (soybean) callus culture, glycol, glycosyl trehalose, gold chloride hcl, halorubrum tebenquichense lipids, heliconia rostrata leaf extract, hesperetin laurate, 1,2,6-hexanetriol, hexylglycerin, hexyl 2-glyceryl ascorbate, hexyl 3-glyceryl ascorbate, hibbertia scandens leaf extract, hibiscus rosa-sinensis flower extract, honey, human fibroblast conditioned media, human fibroblast induced pluripotent cell culture conditioned media, hydrogenated castor oil/sebacic acid copolymer caprate/caprylate, hydrogenated honey, hydrogenated isocetyl olivate, hydrogenated panax ginseng root oil, hydrogenated starch extract, hydrogenated starch hydrolysate, hydrolyzed chicken cartilage extract, hydrolyzed citrus sphaerocarpa peel extract, hydrolyzed cockscomb extract, hydrolyzed corn starch hydroxyethyl ether, hydrolyzed fish scale extract, hydrolyzed hydroxypropyl starch octenylsuccinate, hydrolyzed moms alba leaf extract, hydrolyzed nettle leaf extract, hydrolyzed rye phytoplacenta extract, hydrolyzed starch, hydrolyzed trigonella foenum-graecum seed extract, hydrolyzed wheat protein/peg-20 acetate copolymer, hydroxyethylcellulose/phosphorylcholine glycol acrylate copolymer, hydroxyethyl urea, hydroxyisoleucine, hydroxylated 2-octanol, hydroxypropyl bis-hydroxyethyldimonium chloride, hydroxypropyl oxidized starch pg-trimonium chloride, hydroxypropyltrimonium ascorbate, hydroxypropyltrimonium corn/wheat/soy amino acids, hydroxypropyltrimonium hyaluronate, impatiens balsamina flower/leaf/stem extract, inositol, isopropyl pca, isostearyl acetyl glutaminate, jojoba amino acids, kniphofia uvaria seed oil, kunzea pomifera seed oil, lactic acid, lactitol, lactobacillus/aloe leaf/angelica keiskei/brassica oleracea acephala leaf/carrot leaf/carrot root/cucumber fruit/grapefruit fruit/lavander leaf/melissa officinalis leaf/rosemary leaf/tomato fruit ferment filtrate, lactobacillus/date fruit ferment extract, lactobacillus/horse fat ferment filtrate, lactobacillus/saccharomyces/molasses/calcium carbonate/yeast extract ferment filtrate, lactobacillus/saccharomyces/rhodopseudomonas/molasses/calcium carbonate/yeast extract ferment filtrate, lactobacillus/turmeric ferment filtrate, laetiporus sulphureus mycelium/salvia plebeia extract ferment filtrate, larix leptolepis bark extract, lauramidobutyl guanidine acetate, lauramidobutyl guanidine hcl, laur/myrist/palmitamidobutyl guanidine acetate, lauryl 2-glyceryl ascorbate, 3-laurylglyceryl ascorbate, lauryl 3-glyceryl ascorbate, lauryl malamide, 3-lauryl/tridecylglyceryl ascorbate, lavandula angustifolia (lavender) flower/leaf/stem oil, lavandula angustifolia (lavender) flower/leaf/stem water, lepidium meyenii root powder, lilium tigrinum root extract, lonicera caerulea fruit juice, lonicera caerulea fruit water, ludwigia octovalvis extract, lycium chinense flower extract, lysophosphatidic acid, lysophosphatidylethanolamine, mallotus philippensis fruit extract, malpighia emarginata (acerola) seed extract, maltitol, maltooligosyl glucoside, maltose, mannitol, mannose, meadowfoamamidopropyl betaine, menthyl nicotinate, methoxy peg-7, methoxy peg-10, methoxy peg-16, methoxy peg-25, methoxy peg-40, methoxy peg-100, methoxy peg-113 ascorbic acid, methoxy peg-100/epsilon caprolactone/sebacate copolymer, methoxy peg-13 ethyl polysilsesquioxane, methoxy peg-100/polyepsilon caprolactone, methylamido cellulose gum, methyl carboxymethylphenyl aminocarboxypropylphosphonate, momordica charantia fruit extract, monascus pilosus (mushroom) ferment filtrate, myrciaria dubia seed extract, myristamidobutyl guanidine acetate, myrist/palmitamidobutyl guanidine acetate, myristyl 2-glyceryl ascorbate, myristyl 3-glyceryl ascorbate, myrothamnus flabellifolia extract, octyldodecyl citrate crosspolymer, oleoyl methylglucamide oleate, oroxylum indicum seed extract, oxidized starch hydroxypropyltrimonium chloride, paecilomyces japonica mycelium/ginseng root ferment extract filtrate, paecilomyces japonica/panax ginseng root/juglans regia seed/soybean seed/pinus koraiensis seed ferment extract filtrate, palmitamidobutyl guanidine acetate, n-palmitoyl laurylacetylmethyl aminopropanediol, pastinaca sativa root extract, peg-4, peg-6, peg-7, peg-8, peg-9, peg-10, peg-12, peg-14, peg-16, peg-18, peg-20, peg-32, peg-33, peg-40, peg-45, peg-55, peg-60, peg-75, peg-80, peg-90, peg-100, peg-135, peg-150, peg-180, peg-200, peg-220, peg-240, peg-800, peg-15 butanediol, peg-6 dimethicone, peg-3 methyl ether, peg-4 methyl ether, peg-8 methyl ether dimethicone, peg-5 pentaerythrityl ether, peg-15 pentaerythrityl tetra(laureth-6 carboxylate), peg-90/polyepsilon caprolactone, peg/ppg-28/21 acetate dimethicone, peg/ppg-23/23 butyl ether dimethicone, peg/ppg-24/18 butyl ether dimethicone, peg/ppg-6/4 dimethicone, peg/ppg-60/11 glycerin, pentaerythritol, pentaisostearoyl 3-glyceryl ascorbate, pentapeptide-2, persea thunbergii leaf/root/stem extract, phellinus linteus (mushroom) ferment filtrate, phenoxyethyl pca, phyllostachys edulis extract, phytin, phytosteryl rice branate, pichia ferment lysate filtrate, polydimethylsiloxy ppg-13 butyl ether silsesquioxane, polygamma-glutamic acid, polyglycerin-20, polyglycerin-40, polyglyceryl sorbitol, rh-polypeptide-7, sh-polypeptide-6, sh-polypeptide-8, polyquaternium-64, polyquaternium-65, polyquaternium-10/phosphorylcholine glycol acrylate copolymer, porphyridium polysaccharide, porphyridium polysaccharide hydroxypropyltrimonium chloride, potassium behenoyl hydrolyzed rice protein, potassium dextrin octenylsuccinate, potassium pca, potassium stearoyl hydrolyzed rice protein, potassium succinate, ppg-12 butyl ether dimethicone, ppg-20 diglyceryl ether, ppg-8 glyceryl ether, ppg-6-sorbeth-245, ppg-6-sorbeth-500, ppg-2 tocophereth-5, ppg-20 tocophereth-5, propylene glycol, propylene glycol hyaluronate, prunus amygdalus dulcis (sweet almond) flower extract, prunus mume fruit extract, pseudoalteromonas ferment extract, pseudochoricystis ellipsoidea extract, quercus acutissima wood vinegar extract, resveratryl glucoside, rheum tanguticum root extract, rhizopus/rice ferment extract filtrate, ribose, rosa canina seed extract, rosa odorata extract, saccharomyces/amber powder/honey ferment filtrate, saccharomyces/apple fruit/honey/panax ginseng root/royal jelly/sucrose ferment filtrate, saccharomyces/prunus mume extract ferment filtrate, saccharomyces/stevia rebaudiana leaf/stem ferment extract, saccharomyces/zygosaccharomyces/garlic bulb/(hericum erinaceum/auricularia auricula-judae/grifola frondosa) fruiting body/perilla ocymoides leaf/dioscorea japonica rhizome/(arctium lappa/beet/ginger/ipomoea batatas) root/(phaseolus angularis/sesame/soybean) seed/cladosiphon okamuranus/laminaria japonica/undaria pinnatifida ferment filtrate, saccharum spontaneum extract, schinus molle fruit extract, schizophyllan, schizophyllum commune (mushroom) ferment filtrate, schizophyllum commune mycelium/rice ferment extract filtrate, schizosaccharomyces ferment extract filtrate, schizosaccharomyces ferment filtrate, sea water, sesquioctyldodecyl lauroyl glutamate, sodium acetylated hyaluronate, sodium acetyl ethylcarboxyl methylthiazolidine carboxylate, sodium/aluminum hydroxide/oxalate/sulfate, sodium/aluminum/iron/sulfate/citrate/hydroxide, sodium/aluminum/iron/sulfate/oxalate/hydroxide, sodium/aluminum/iron/sulfate/tartarate/hydroxide, sodium carboxymethyl hyaluronate, sodium dextrin octenylsuccinate, sodium dimyristoyl glyceryl ascorbate, sodium distearoyl glyceryl ascorbate, sodium ethylhexylglyceryl ascorbate, sodium glucuronate, sodium heparosan, sodium hyaluronate crosspolymer-2, sodium hydroxypropyl cyclodextrin octenylsuccinate, sodium hydroxypropyl oxidized starch succinate, sodium oxidized starch acetate/succinate, sodium pca, sodium polygamma-glutamate crosspolymer, sodium trehalose octenylsuccinate, solanum aviculare seed oil, solanum lycopersicum (tomato) fruit, sorbeth-6, sorbeth-20, sorbeth-30, sorbeth-40, sorbitol, sorbityl silanediol, soy protein phthalate, sparassis crispa (mushroom) ferment filtrate, spondias mombin fruit extract, stearyl acetyl glutaminate, stearyl/octyldodecyl citrate crosspolymer, stearyl palmitate, streptococcus zooepidemicus extract, sucrose, tea-dextrin octenylsuccinate, telopea speciosissima flower/leaf extract, terminalia ferdinandiana seed oil, tetrabaena socialis extract, theobroma grandiflorum fruit juice, titanium citrate, tocopheryl retinoate, trehalose, tricholoma magnivelare extract, triglycereth-7 citrate, trimethylamine oxide, trioxaundecanedioic acid, tripropylene glycol, undaria pinnatifida root powder, urea, urea-d-glucuronic acid, vigna angularis seedcoat extract, vitis coignetiae sap, vp/vinyl alcohol copolymer, xylitol, xyloglucan, and xylose.

The personal care composition (202) also includes an abrasive, or exfoliating ingredient. The abrasive removes dead or dying skin particles to reveal healthier looking skin. The abrasive may form 50-70 percent by weight of the personal care composition (202).

Examples of abrasives include, but are not limited to sodium chloride, actinidia chinensis (kiwi) seed, adansonia digitata seed powder, agave americana leaf powder, alexandrite powder, alumina, aluminum iron silicates, aluminum isopropoxide, aluminum silicate, amethyst powder, ammonium acryloyldimethyltaurate/laureth-7 methacrylate copolymer, amorphophallus konjac root powder, ampelopsis japonica root powder, angelica archangelica seed powder, angelica dahurica root powder, angelica dahurica seed powder, arachis hypogaea (peanut) flour, argania spinosa shell powder, artemisia caruifolia powder, astragalus membranaceus root powder, astrocaryum murumuru seed powder, atractylodes macrocephala root/stalk powder, attapulgite, avena sativa (oat) bran, avena sativa (oat) kernel flour, avena sativa (oat) kernel meal, bambusa arundinacea juice, bambusa arundinacea stem powder, bertholletia excelsa seed powder, bletilla striata root/stalk powder, 1,4-butanediol/succinic acid/adipic acid/hdi copolymer, butyrospermum parkii (shea) nut shell powder, calcium carbonate, calcium phosphate, calcium potassium sodium phosphate, calcium pyrophosphate, calcium sulfate, calcium sulfate hydrate, calcium titanium borosilicate, cannabis sativa seedcake, carapa guaianensis seed powder, carya illinoinensis (pecan) shell powder, castanea crenata (chestnut) pellicle powder, castanea crenata (chestnut) shell powder, cellulose, chalk, charcoal powder, chenopodium quinoa seed, chitin, chlorophora tinctoria wood extract, chondrus crispus powder, cicer arietinum seed powder, citrullus lanatus (watermelon) seed powder, citrus nobilis (mandarin orange) peel powder, citrus tangerina (tangerine) peel, cnidium monnieri seed powder, cocos nucifera (coconut) fruit, cocos nucifera (coconut) shell powder, codonopsis pilosula root powder, coffea arabica (coffee) seed powder, coffea robusta seed powder, colloidal oatmeal, conchiolin powder, coptis chinensis root/stalk powder, coral powder, corylus avellana (hazelnut) seed powder, corylus avellana (hazelnut) shell powder, cymbopogon flexuosus leaf powder, diamond powder, diatomaceous earth, dicalcium phosphate, dicalcium phosphate dihydrate, dipentaerythrityl hexaisostearate, dolomite, egg shell powder, eijitsu, elguea clay, emerald, empetrum nigrum flower/fruit/leaf extract, eucalyptus globulus leaf powder, euterpe oleracea pulp powder, euterpe oleracea seed powder, ficus carica (fig) seed, foeniculum vulgare (fennel) seed, fragaria vesca (strawberry) seed, fuller's earth, garcinia mangostana peel powder, garnet powder, glycine soja (soybean) flour, glycine soja (soybean) seed powder, hakka, helianthus annuus (sunflower) seed, helianthus annuus (sunflower) seedcake, helianthus annuus (sunflower) seed flour, hibiscus sabdariffa flower powder, hippophae rhamnoides husk powder, hippophae rhamnoides seed powder, holmium oxide, honey powder, hordeum distichon (barley) seed flour, hordeum vulgare powder, hordeum vulgare seed flour, hydrated silica, hydroxyapatite, 3-hydroxybutyrate/4-hydroxybutyrate copolymer, 3-hydroxybutyrate/3-hydroxyhexanoate copolymer, hydroxybutyric acid/hydroxypentanoic acid copolymer, illite, indium silver zinc diethyldithiocarbamate nitrate, ipomoea hederacea seed powder, isatis tinctoria root powder, jojobamidopropyl betaine, juglans mandshurica (walnut) shell powder, juglans regia (walnut) shell powder, juniperus oxycedrus wood powder, kaolin, kochia scoparia fruit powder, kurumi kaku, lactide, lauryl acrylate/va crosspolymer, leonurus artemisia powder, limonia acidissima shell powder, linum usitatissimum (linseed) seed flour, litchi chinensis seed powder, lithothamnion calcareum powder, lithothamnion corallioides powder, loess, luffa cylindrica fruit, luffa cylindrica fruit powder, lygodium japonicum spore, macadamia integrifolia shell powder, macadamia ternifolia shell powder, magnesium potassium fluorosilicate, magnesium sodium fluorosilicate, magnesium trisilicate, magnolia denudata bud powder, mangifera indica (mango) seed, marble powder, matricaria maritima powder, matteuccia struthiopteris bud powder, mauritia flexuosa pulp powder, melaleuca alternifolia (tea tree) leaf, melaleuca alternifolia (tea tree) leaf powder, methacryloyloxypropyl polysilsesquioxane, microcrystalline cellulose, momordica charantia leaf extract, momordica charantia leaf powder, montmorillonite, moringa oleifera fruit powder, moroccan lava clay, mother of pearl, mudstone powder, myristyl betaine, nacre powder, nannochloropsis oculata powder, nelumbo nucifera flower powder, nelumbo nucifera seed powder, nigella damascena seed powder, oenothera biennis (evening primrose) seed, olea europaea (olive) fruit, olea europaea (olive) husk powder, olea europaea (olive) leaf powder, olea europaea (olive) seed, olea europaea (olive) seed powder, oryza sativa (rice) bran, oryza sativa (rice) germ powder, ostrea shell extract, ostrea shell powder, oubaku, ovum powder, oyster shell powder, paeonia lactiflora root powder, paeonia officinalis flower powder, papaver rhoeas seed, papaver somniferum seed, passiflora edulis seed powder, pegmatite, perlite, persea gratissima (avocado) fruit powder, persea gratissima (avocado) seed, phaseolus angularis seed, phaseolus radiatus seed starch, phyllostachys edulis stem powder, pinus pinaster bark powder, pistacia vera shell powder, plantago psyllium husk, plantago psyllium husk powder, platinum powder, polyethylene, polyhydroxybutyrate, polylactic acid, poria cocos powder, porphyra umbilicalis powder, potassium undecylenoyl glutamate, prunus amygdalus dulcis (sweet almond) seedcoat powder, prunus amygdalus dulcis (sweet almond) seed meal, prunus amygdalus dulcis (sweet almond) shell powder, prunus armeniaca (apricot) seed powder, prunus avium (sweet cherry) seed powder, prunus avium (sweet cherry) shell powder, prunus cerasus (bitter cherry) shell powder, prunus mume fruit, prunus mume seed powder, prunus persica (peach) flower powder, prunus persica (peach) seed powder, pumice, punica granatum seed, punica granatum seed powder, quartz, quartz powder, quassia amara wood powder, quercus suber bark, rosa canina seed, rosa canina seed powder, rosa centifolia flower powder, rosa rugosa bud powder, rubus idaeus (raspberry) seed, rubus idaeus (raspberry) seed powder, salt mine mud, salvia hispanica seed, salvia hispanica seed powder, sand, sanguisorba officinalis root/stalk powder, schinziophyton rautanenii kernel powder, schist, sclerocarya birrea seed powder, scutellaria baicalensis root powder, sea salt, secale cereale (rye) seed flour, sedimentary rock powder, silica, silkworm frass, sillimanite, siraitia grosvenorii fruit powder, smilax lancefolia root/stalk powder, sodium/aluminum hydroxide/oxalate/sulfate, sodium/aluminum/iron hydroxide/oxalate/sulfate, sodium bicarbonate, sodium calcium boron phosphate, sodium hydroxypropyl starch phosphate, sodium magnesium fluorosilicate, sodium silicoaluminate, solum diatomeae, sophora flavescens root powder, sponge, starch laurate, stemona sessifolia root powder, symphytum officinale leaf powder, synthetic ruby, synthetic ruby powder, syringa oblata bark/leaf powder, talc, theobroma cacao (cocoa) husk, theobroma cacao (cocoa) seed powder, theobroma cacao (cocoa) shell powder, theobroma grandiflorum seed powder, thuja orientalis branch/leaf powder, tin oxide, titanium oxynitride, topaz, touki, tribulus terrestris fruit powder, tricalcium phosphate, trichosanthes kirilowii fruit powder, triticum vulgare (wheat) bran, triticum vulgare (wheat) germ powder, triticum vulgare (wheat) kernel flour, triticum vulgare (wheat) starch, tungsten oxide, vaccinium angustifolium (blueberry) seed, vaccinium macrocarpon (cranberry) seed, vaccinium macrocarpon (cranberry) seed powder, vanilla planifolia seed powder, vanilla tahitensis seed, viola prionantha powder, virola sebifera seed powder, vitis vinifera (grape) seed powder, volcanic ash, volcanic rock, wollastonite, wood powder, yokuinin, yttrium oxide, zea mays (corn) cob meal, zea mays (corn) cob powder, zea mays (corn) kernel meal, zea mays (corn) seed flour, zea mays (corn) starch, and zirconium silicate.

The personal care composition (202) also includes a cleanser or foaming agent that cleans the surface. The cleanser serves to remove dirt or other particles from the surface of the skin. The cleanser may form 1-3 percent by weight of the personal care composition (202).

Examples of cleansers include, but are not limited to polysorbate surfactant, diglyceryl sorbitan tetraethylhexanoate, disodium hydroxydecyl sorbitol citrate, glyceryl/sorbitol oleate/hydroxystearate, ozonized polysorbate 80, peg-20 sorbitan cocoate, peg-40 sorbitan diisostearate, peg-2 sorbitan isostearate, peg-5 sorbitan isostearate, peg-20 sorbitan isostearate, peg-40 sorbitan lanolate, peg-75 sorbitan lanolate, peg-10 sorbitan laurate, peg-40 sorbitan laurate, peg-44 sorbitan laurate, peg-75 sorbitan laurate, peg-80 sorbitan laurate, peg-3 sorbitan oleate, peg-6 sorbitan oleate, peg-20 sorbitan oleate, peg-40 sorbitan oleate, peg-80 sorbitan palmitate, peg-40 sorbitan perisostearate, peg-40 sorbitan peroleate, peg-3 sorbitan stearate, peg-4 sorbitan stearate, peg-6 sorbitan stearate, peg-40 sorbitan stearate, peg-60 sorbitan stearate, peg-30 sorbitan tetraoleate, peg-40 sorbitan tetraoleate, peg-60 sorbitan tetraoleate, peg-60 sorbitan tetrastearate, peg-4 sorbitan triisostearate, peg-20 sorbitan triisostearate, peg-160 sorbitan triisostearate, peg-2 sorbitan trioleate, peg-3 sorbitan tristearate, polyglyceryl-2 sorbitan tetraethylhexanoate, polysorbate 20, polysorbate 21, polysorbate 40, polysorbate 60, polysorbate 61, polysorbate 65, polysorbate 80, polysorbate 81, polysorbate 85, polysorbate 80 acetate, polysorbate 20 dicinnamate, polysorbate 80 dicinnamate, ppg-8 sorbitol castor oil, sorbeth-2 beeswax, sorbeth-6 beeswax, sorbeth-8 beeswax, sorbeth-20 beeswax, sorbitan caprylate, sorbitan cocoate, sorbitan diisostearate, sorbitan dioleate, sorbitan distearate, sorbitan fatty acid ester, sorbitan isostearate, sorbitan laurate, sorbitan oleate, sorbitan olivate, sorbitan palmate, sorbitan palmitate, sorbitan sesquicaprylate, sorbitan sesquiisostearate, sorbitan sesquioleate, sorbitan sesquistearate, sorbitan stearate, sorbitan theobroma grandiflorum seedate, sorbitan triisostearate, sorbitan trioleate, sorbitan tristearate, and sorbitan undecylenate.

The personal care composition (202) also includes a dispersing agent to suspend the cleanser in the personal care composition (202). The dispersing agent may form from 0.1 to 2 percent by weight of the personal care composition (202).

Examples of dispersing agents include, but are not limited to hydrophilic fumed silica, acrylates copolymer, acrylates/methoxy peg-15 methacrylate copolymer, acrylates/steareth-20 methacrylate crosspolymer, acrylates/vinyl isodecanoate crosspolymer, acrylates/vp copolymer, acrylic acid/acrylamidomethyl propane sulfonic acid copolymer, acrylic acid/stearyl methacrylate/dimethicone methacrylate copolymer, acrylic acid/vp crosspolymer, ammonium acryloyldimethyltaurate/carboxyethyl acrylate crosspolymer, ammonium oleoyl/palmitoyl glycerophosphoethylamido glutaroyl oligopeptide-114, ammonium styrene/acrylates copolymer, ammonium va/acrylates copolymer, barium/calcium/silicon/titanium oxide, bentonite, benzalkonium montmorillonite, benzalkonium sepiolite, biotite, bis-peg-10 dimethicone, bis-peg-1 dimethicone/ppg-26/ipdi copolymer, butyl babassuate, calcium lignosulfonate, calcium polyglutamate crosspolymer, c4-24 alkyl dimethicone/divinyldimethicone crosspolymer, chitosan lauramide succinamide, c18-50 isoparaffin, cobalt dna, corallina officinalis powder, corn starch/acrylamide/sodium acrylate copolymer, c6-14 perfluoroalkylethyl acrylate/hema copolymer, dehydroxanthan gum, diallyloxyneohexyl zirconium tridecanoate, dihydrogenated tallow benzylmonium hectorite, dimer dilinoleyl dimer dilinoleate/ipdi copolymer, dimethicone crosspolymer, dimethiconol/stearyl methicone/phenyl trimethicone copolymer, dimethylol urea/phenol/sodium phenolsulfonate copolymer, dipentaerythrityl pentaisostearate, disodium methylene dinaphthalenesulfonate, disteardimonium hectorite, ditrimethylolpropane isostearate/sebacate, ditrimethylolpropane triethylhexanoate, erythrityl triethylhexanoate, ethylene/ma copolymer, ethylene/va copolymer, ethylhexyl hydroxystearoyl hydroxystearate, ethyl trisiloxane, feruloyl soy glycerides, glass, glass beads, hdi/pei-45/smdi crosspolymer, hea/sodium acryloyldimethyltaurate/steareth-20 methacrylate copolymer, hectorite, hydrogenated isocetyl olivate, hydrogenated lecithin, hydrolyzed defatted soybean seed, hydrolyzed va/vinyl acetoacetate copolymer, hydroxyethyl acrylate/sodium acryloyldimethyl taurate copolymer, hydroxyethyl pei-1000, hydroxyethyl pei-1500, hydroxypropyl starch, hydroxypropyltrimonium maltodextrin crosspolymer, isobornyl dipropylene glycol ethers, isobutylene/ma copolymer, isopropyl babassuate, isopropyl ester of pvm/ma copolymer, isotridecylcarbamatotolyl aminohexylurea, magnesium phosphate, maltodextrin, methacryloyl ethyl betaine/acrylates copolymer, methoxy peg-17/dodecyl glycol copolymer, methoxy peg-22/dodecyl glycol copolymer, methoxy peg-114/polyepsilon caprolactone, methoxy peg-100/polyepsilon caprolactone ethylhexanoate, methoxy peg-100/polyepsilon caprolactone palmitate, methyl methacrylate, myristoyl/pca chitin, nitrocellulose, octyldodecyl/ppg-3 myristyl ether dimer dilinoleate, peg-18 castor oil dioleate, peg-150/decyl alcohol/smdi copolymer, peg-12 dimethicone crosspolymer, peg-150/stearyl alcohol/smdi copolymer, pei-7, pei-10, pei-15, pei-30, pei-35, pei-45, pei-250, pei-275, pei-700, pei-1000, pei-1400, pei-1500, pei-1750, pei-2500, pei-14m, pentafluoropropane, perfluorononyl octyldodecyl glycol meadowfoamate, perlite, phosphonobutanetricarboxylic acid, polyacrylamidomethylpropane sulfonic acid, polyacrylate-10, polyacrylate-11, polyacrylate-27, polyacrylate crosspolymer-4, polyacrylate crosspolymer-5, polyacrylate crosspolymer-10, polyacrylate crosspolymer-11, polyacrylate crosspolymer-14, polycaprolactone, polyester-16, polyester-24, polyethylacrylate, polyglyceryl-4 isostearate/laurate, polyhydroxystearamidopropyl dimethylamine, polyhydroxystearic acid, polyoxymethylene cyanoguanidine urea, polyperfluoroethoxymethoxy peg-2 phosphate, polyquaternium-83, polyquaternium-102, polyquaternium-103, polyurea crosspolymer-1, polyurethane-59, polyvinyl imidazolinium acetate, polyvinyl methyl ether, ppg-3 myristyl ether neoheptanoate, propylene glycol ricinoleate, pvm/ma copolymer, pvp, pvp/va/itaconic acid copolymer, quaternium-18 bentonite, quaternium-18/benzalkonium bentonite, quaternium-18 hectorite, quaternium-90 bentonite, quaternium-90 montmorillonite, quaternium-90 sepiolite, rhizobian gum, silica, silica caprylyl silylate, silica cetyl silylate, silica dimethicone silylate, silica dimethyl silylate, silica silylate, sodium acrylate/acryloyldimethyltaurate/dimethylacrylamide crosspolymer, sodium acrylates/beheneth-25 methacrylate crosspolymer, sodium acrylate/sodium acryloyldimethyl taurate/acrylamide copolymer, sodium acrylate/sodium acryloyldimethyl taurate copolymer, sodium acrylates/vinyl isodecanoate crosspolymer, sodium acrylic acid/ma copolymer, sodium acryloyldimethyl taurate/acrylamide/vp copolymer, sodium c4-12 olefin/maleic acid copolymer, sodium dextran sulfate, sodium dimaltodextrin phosphate, sodium glycereth-1 polyphosphate, sodium isooctylene/ma copolymer, sodium magnesium fluorosilicate, sodium polyglutamate crosspolymer, starch hydroxypropyltrimonium chloride, stearalkonium bentonite, stearalkonium hectorite, stearyl/ppg-3 myristyl ether dimer dilinoleate, stearylvinyl ether/ma copolymer, styrene/acrylates/acrylonitrile copolymer, styrene/acrylates/ammonium methacrylate copolymer, styrene/ma copolymer, sucrose benzoate/sucrose acetate isobutyrate/butyl benzyl phthalate copolymer, sunflower seed oil ethyl ferulate esters, synthetic ruby, synthetic ruby powder, tosylamide/epoxy resin, tosylamide/formaldehyde resin, tribenzoyl triricinolein, vinyl alcohol/vinylformamide copolymer, vp/dimethylaminoethylmethacrylate copolymer, vp/eicosene copolymer, vp/hexadecene copolymer, and vp/va copolymer.

In addition to the ingredients mentioned above, the composition (202) may include other ingredients to improve the application aesthetics, contact aesthetics, or other characteristics of the composition (202). For example, the composition (202) may include at least one of a fragrance, a colorant, a structurant, and an emulsifier.

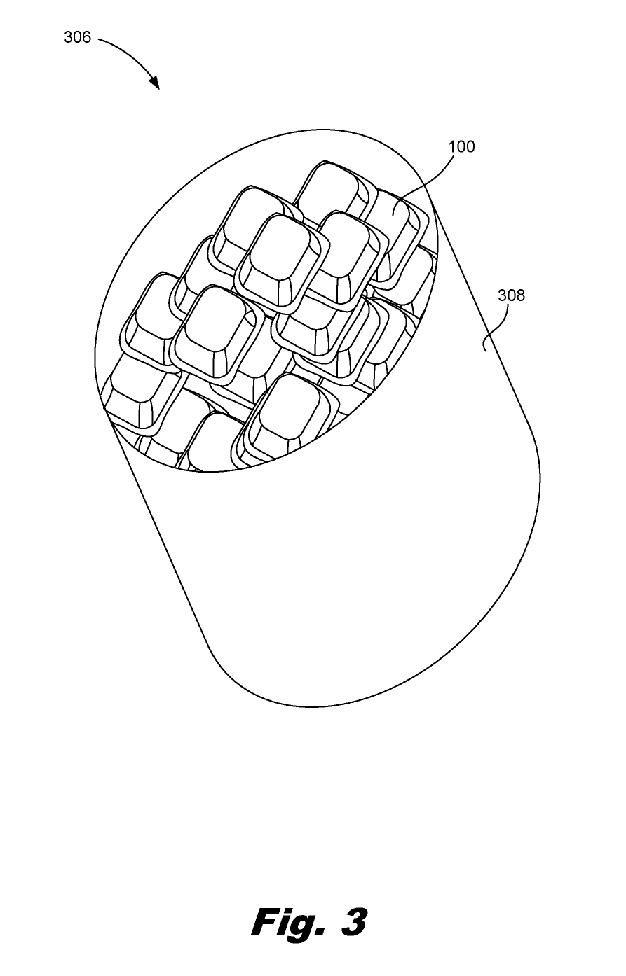

FIG. 3 is a diagram of an example of a personal care consumer product (306) that includes multiple personal care devices (100) according to the principles described herein. The product (306) includes a housing (308) that holds multiple personal care devices (100). For simplicity, in FIG. 3, just one personal care device (100) is referenced with a number. As described above, the personal care devices (100) are disposed within the housing (308) and include an enclosed film that is dissolvable in water and a personal care composition (FIG. 2, 202) that is disposed within the enclosed film. The personal care composition (FIG. 2, 202) is deposited on a surface when the enclosed film dissolves. In some examples, the product (306) includes a protective cover such as a lid. During use, the lid of the housing (308) is removed and the composition (FIG. 2, 202) exposed for application to a surface. When not in use, the lid of the housing (306) is replaced such that the devices (10) can be cleanly transported without depositing the composition (202) on any bag or other transport device that may be holding the container (100) and composition (202).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cleansing device comprising:
a container dissolvable in water; and
a personal care composition disposed within the container, wherein the personal care composition is to be deposited on a surface when the container dissolves.

2. The device of claim 1, wherein the personal care composition comprises between 10-30 percent by weight of an oil.

3. The device of claim 1, wherein the container is formed of a polyvinyl alcohol film.

4. The device of claim 1, wherein the container is formed of a polyvinyl acetate film.

5. The device of claim 1, wherein the personal care composition is a skin care composition.

6. The device of claim 1, wherein the personal care composition is a body scrub.

7. The device of claim 1, wherein the container is sized to hold a single-use amount of the personal care composition.

8. A cleansing device comprising:
a polyvinyl alcohol container that is dissolvable when in contact with water;
a personal care composition disposed within the polyvinyl alcohol container that is deposited on skin when the polyvinyl alcohol container dissolves, wherein the personal care composition comprises:
a carrier fluid;
a humectant;
an abrasive;
a cleanser; and
a dispersing agent.

9. The device of claim 8, wherein the personal care composition is a body scrub.

10. The device of claim 8, wherein the carrier fluid forms from 10-30 percent by weight of the personal care composition.

11. The device of claim 8, wherein the humectant forms 20-30 percent by weight of the oil-based personal care composition.

12. The device of claim 8, wherein the cleanser forms from 1-3 percent by weight of the personal care composition.

13. The device of claim 8, further comprising at least one of a fragrance, a colorant, a structurant, and an emulsifier.

14. A personal care consumer product comprising:
a housing to hold multiple personal care devices;
multiple personal care devices disposed within the housing, wherein a personal care device comprises:
an enclosed film dissolvable in water; and
a personal care composition disposed within the enclosed film, wherein the personal care composition is to be deposited on a surface when the enclosed film dissolves.

15. The product of claim 14, wherein the personal care composition comprises between 10 and 30 percent of an oil.

16. The product of claim 15, wherein the composition is a skin scrub.

17. The product of claim 15, wherein the composition is at least one of a face scrub, a foot scrub, a hand scrub, a body scrub, an acne treatment, a soap, and a lotion.

18. The product of claim 15, wherein the enclosed film is at least one of a polyvinyl alcohol film and a polyvinyl acetate film.

19. The product of claim 15, wherein the personal care composition includes a surfactant.

20. The device of claim 8, further comprising at least one of a fragrance, a colorant, a structurant, and an emulsifier.
